(19) United States
(12) Patent Application Publication
(10) Pub. No.: US 2007/0135761 A1
(43) Pub. Date: Jun. 14, 2007

(54) Breast pump

(76) Inventors: Kai-sheng Cheng, Beavercreek, OH (US); Kendra Rose, Celina, OH (US); Viresh Bijawat, Centerville, OH (US); Jonathan W. Conaway, Liberty Township, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 11/298,906

(22) Filed: Dec. 9, 2005

(51) Int. Cl. A61M 1/06

(52) U.S. Cl. 604/74

(57) ABSTRACT

A breast pump assembly comprising: (a) a breast milk caddy including a frame having an arm repositionable with respect to a handle between a folded position and an extended position, and a milk extraction device repositionably mounted to the arm of the frame, the milk extraction device including complementary top and bottom components removably mounted to one another, the bottom component including a breast milk extraction vessel, and the top component including a breast cone having an inlet for receiving a woman's breast and an outlet in communication with the extraction vessel for storing extracted milk, the top component also including a conduit for delivering suction to the inlet of the breast cone; (b) a breast pump including a fluid pump including an inlet and an outlet, where the fluid pump generates suction at the inlet during operation of the fluid pump, and a control system in electrical communication with the fluid pump for controlling operation of the fluid pump, the control system including a soft start logic to gradually increase the suction generated by the fluid pump; and (c) tubing mounted in fluid communication with the fluid pump and the milk extraction device for delivering the generated suction from the fluid pump and to the conduit of the milk extraction device.

### FIELD OF THE INVENTION

The present invention is directed to hardware utilized to effectuate the pumping of breast milk.

### BRIEF INTRODUCTION TO THE INVENTION

The present invention includes numerous novelties relating to hardware utilized to effectuate the pumping of breast milk. One exemplary novelty of the present invention includes a breast pump having a visual display for providing indicia to a user regarding various operational conditions such as, without limitation, current usage time, daily usage time, aggregate usage time, speed of the pump, current time and date, and the distribution of suction applied to each connected tube. The exemplary breast pump also includes a soft start feature that gradually ramps up the suction applied to one or both breasts to provide greater comfort to the user.

Portability of the exemplary breast pump is an important consideration. An on-board retractable power cord allows users of the instant pump to quickly withdraw the power cord prior to use and correspondingly retract the cord when pumping operations have ceased. The instant breast pump includes an adapter at the end of the retractable power cord that receives a universal male power projection. In this manner, AC/DC converters, cigarette lighter adapters, and battery packs may all be used to power the exemplary breast pump so long as these devices include the universal male power projection. An insulated carrier may be included with the exemplary breast pump that includes cargo area for storing the breast pump itself and one or more hardware items for use with the breast pump or breast feeding operations. Exemplary hardware includes, without limitation, AC/DC converters, cigarette lighter adapters, battery packs, tubing, refrigerated or frozen packs, extraction horns, breast milk cups or bottles, breast milk extraction assemblies, and holders for breast milk extraction assemblies. For example, the tubing may be wrapped around a spindle to facilitate quick deployment and storage subsequent to use.

Exemplary holders for breast milk extraction assemblies include two repositionable arms that extend outward from a handle portion. Each repositionable arm includes a track along which an exemplary breast milk extraction assembly is repositionably mounted so that each breast milk extraction assembly can be laterally spaced to correspond to the space between the nipples of adjacent breasts. The arms of the holder are adapted to be repositioned for storage to decrease the area occupied by the holder. In one exemplary embodiment, each arm includes an actuator that allows the arm to move from an extended position to a retracted position. However, the invention contemplates that the actuator may be partially or wholly removed from each arm.

The aforementioned samples of the exemplary novelties associated with the present invention is not a complete listing or summary of the instant invention. Reference is had to the remainder of the instant application for a more thorough discussion of the present invention. It is to be understood that this introduction to the invention is not intended to, and does not, restrict the scope of the invention.

### BRIEF DESCRIPTION OF THE DRAWINGS

FIG. 1 is a perspective view of an exemplary breast pump assembly in accordance with the present invention;

FIG. 2 is a frontal view of an exemplary control panel, and a perspective view of two exemplary extraction assemblies and an exemplary extraction vessel holder, showing tubing being run between the panel and extraction assembly;

FIG. 3 is a perspective view of two exemplary extraction assemblies mounted to an exemplary extraction vessel holder;

FIG. 4 is a perspective view of two exemplary extraction assemblies dismounted from an exemplary extraction vessel holder;

FIG. 5 is a perspective view of an exemplary extraction vessel holder having two exemplary retention rings mounted thereto;

FIG. 6 is an exploded view of the exemplary extraction vessel holder and two exemplary retention rings of FIG. 5;

FIG. 7 is a frontal view of an exemplary extraction vessel holder having extendable arms in the folded position;

FIG. 8 is an exploded view of an exemplary extraction assembly;

FIG. 9 is an elevated perspective view of an exemplary breast pump;

FIG. 10 is an exploded view of the exemplary breast pump of FIG. 9;

FIG. 11 is an exploded view of an exemplary diaphragm module of the exemplary breast pump of FIG. 10;

FIG. 12 is a frontal view of an exemplary control panel of the exemplary breast pump of FIG. 10;

FIG. 13 is a decision tree for a soft start routine of the exemplary breast pump of FIG. 10;

FIG. 14 is an exploded view of an exemplary power module of the exemplary breast pump of FIG. 10; and

FIG. 15 is a perspective view showing exemplary hardware for use with the exemplary breast pump of FIG. 10.

### DETAILED DESCRIPTION

The exemplary embodiments of the present invention are described and illustrated below to encompass equipment for use in extracting breast milk from one or both breasts of a human being. Of course, it will be apparent to those of ordinary skill in the art that the embodiments discussed below are exemplary in nature and may be reconfigured without departing from the scope and spirit of the present invention. However, for clarity and precision, the exemplary embodiments as discussed below may include optional steps, methods, and features that one of ordinary skill should recognize as not being a requisite to fall within the scope of the present invention.

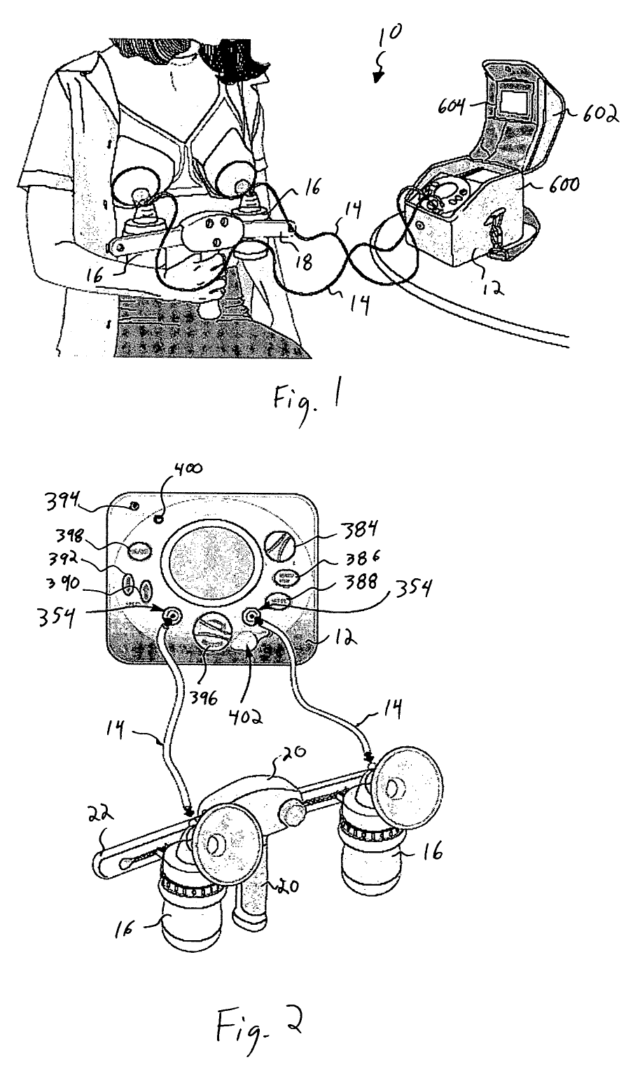

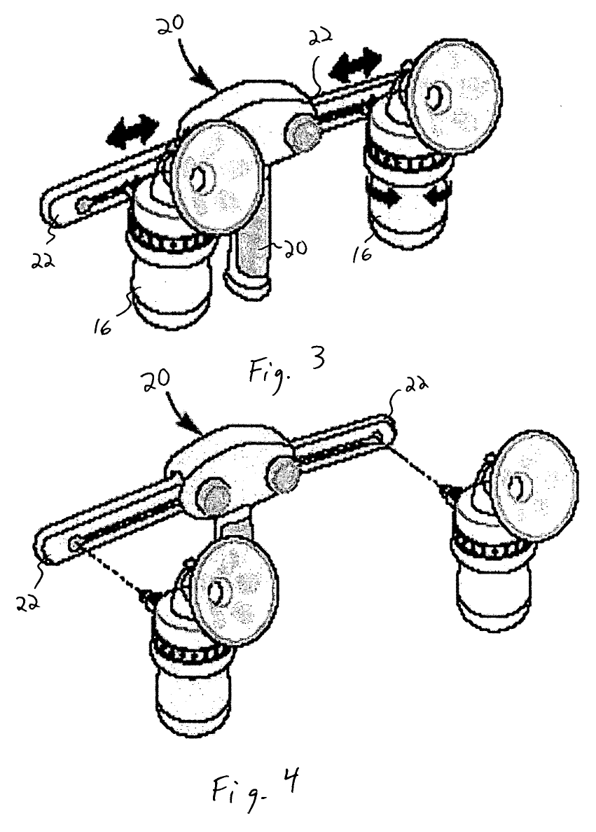

Referencing FIGS. 1 and 2, a breast pump system 10 includes a breast pump 12, tubing 14, extraction assemblies 16, and an extraction vessel holder 18. In operation, the tubing is connected to one or more extraction assemblies 16 and concurrently to the breast pump 12. The extraction vessel holder 18 retains the extraction assemblies 16 in relative position to allow the extraction process to be carried out.

Referring to FIGS. 2-7, an exemplary extraction vessel holder 18 includes a handle portion 20 having two repositionable arms 22. Each repositionable arm 22 is adapted to receive a retention ring 24 of the extraction assembly 16.

Complementary parts 26, 28 mount to one another to define the handle portion 20. The first part 26 includes a vertically extending handle 30 integral with a top housing 32. The vertically extending handle 30 is fabricated to include a protruding hump 34 adjacent to a handle grip 36. The vertically extending handle 30 is fabricated from a first polymer and the handle grip 36 is fabricated from a second, more resilient polymer. In this exemplary embodiment, the vertically extending handle 30 includes a trench into which the second, more resilient polymer is injection molded to form the grip 36 so that the edge of the grip is substantially flush with the adjacent edge of the vertically extending handle 30. The top housing 32 of the first part 26 includes two orifices 38, 40 spaced apart from one another that will accommodate actuators 42 of the repositionable arms 22. Corresponding grooves 44 are cut out of the top housing 32 to allow for a ninety degree range of movement for the repositionable arms 22.

The second part 28 of the handle portion 20 includes a vertically extending handle 48 integral with a top housing 50. The vertically extending housing 50 is fabricated to include a protruding hump 52 that cooperates with the protruding hump 34 of the first part 26 when the handle portion 20 is assembled to form a circumferential stop 54. The second part 28 includes five through holes 56 that are adapted to be aligned with five receivers of the first part 26. Five screws are inserted through the holes 56 and received by the receivers of the first part 26 to mount the first and second parts 26, 28 to each other.

An interior portion 60 of the top housing 50 is inset and includes a pair of raised circular walls 62 at generally the same height as the adjacent wall 64. The adjacent wall 64 height is reduced or cut-out to accommodate pivotal motion of the repositionable arms 22. Within each raised circular wall 62 is a diamond-shaped raised wall 66. Two corners 68 of the diamond-shaped raised wall 66 opposite one another are substantially perpendicular, whereas the other opposite corners 70 are substantially rounded. As will be discussed in more detail hereafter, the interior of the diamond-shaped raised wall 66 receives a diamond-shaped projection 72 of each actuator 42.

Each repositionable arm 22 includes five components. The first two components are complementary halves 74, 76 that form the infrastructure of the arm 22. The first half 76 comprises an elongated section 78 integrated into a circular end 80. The elongated section 78 includes a longitudinal groove 82 having a circular opening 84 that accepts a portion of the retention ring 24. The interior 86 of the first half 74 includes a plurality of teeth 88 in a spaced apart manner running along each side of the longitudinal groove 82. The exterior of the circular end 80 adjacent to the longitudinal groove 82 includes a floor 90 with a raised circular ring 92 circumferentially spaced outward from an orifice 94.

The second half 74 of each repositionable arm 22 also includes an elongated section 96 integral with a circular end 98. Approximate the end of the elongated section 96 is a through hole 100 adapted to receive a screw utilized to mount the halves 74, 76 to one another. Each circular end 98 includes a cylindrical inset cavity 106. The base of the cavity 106 includes an orifice 108 outlined by an hourglass shaped planar floor. Two projections 112 extend upward from the floor, with each projection having a top surface that increases in slope moving in the clockwise direction. The interior of the circular end 98 includes a projection that mirrors the topography on the inset cavity 106 so that the hourglass portion 112 extends farther than the two adjacent areas. A pair of raised rails 126 extend longitudinally along the interior of the second half 74 and are aligned with the longitudinal groove 82 of the first half 76 upon mounting of the halves to one another.

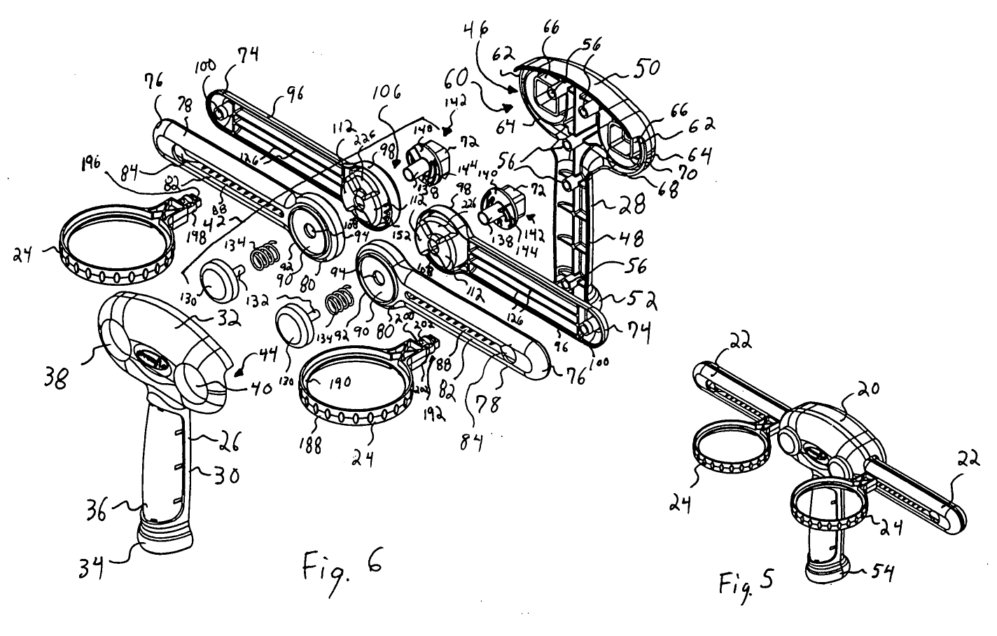

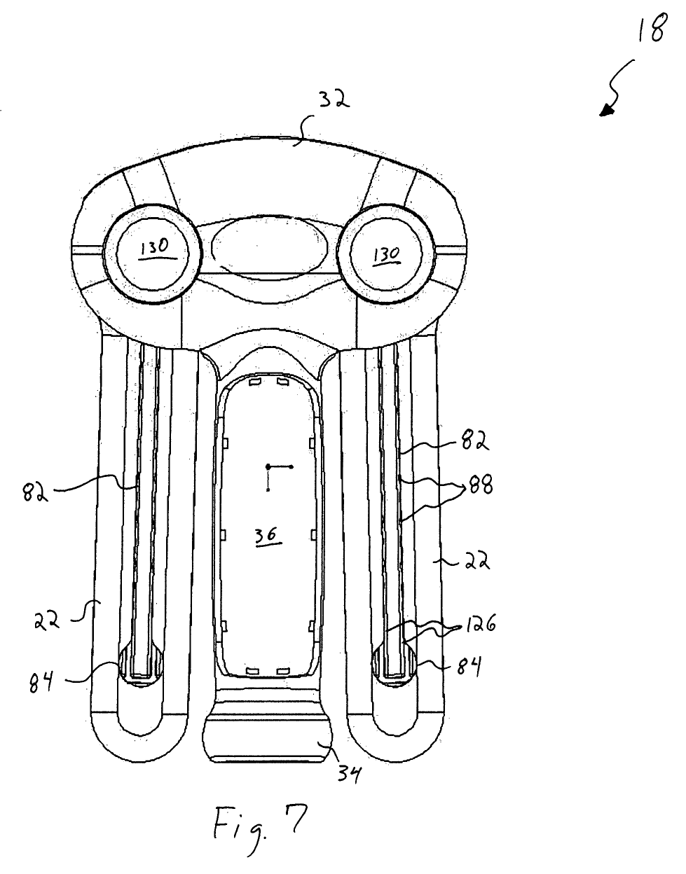

Each repositionable arm 22 includes an actuator 42 cooperating with the diamond-shaped raised wall 66 and circular end 98 to reposition each arm 22 between a folded and unfolded position (Compare FIGS. 5 and 7). Each actuator 42 includes a button 130 having a post 132 circumscribed by a spring 134. The spring 134 is retained in the circumscribed manner by a four angle braces. The post 132 is also partially circumscribed by a hollow cylinder 138 extending from a circular platform 140. The cylinder 138 and platform are integrated into a cam member 142 having cam projections 144 partially circumscribing the cylinder 138. A diamond-shaped projection 72 extends from the underside of the platform 140 and is adapted to be seated within the opening of the diamond-shaped raised wall 66. The diamond-shaped projection 72 includes two corners that are rounded and two corners that are perpendicularly angled to correspondingly fit within the contours of the diamond-shaped raised wall 66.

Assembly of each repositionable arm 22 includes mounting the halves 74, 76 to one another. The interiors of each elongated section 78, 96 and the interiors of each circular end 80, 98 are oriented to face and abut one another as shown in FIG. 5. A flange on the interior of the circular end 80 interfaces with a series of teeth 152 on the interior of the other circular end 98 to mount the ends 80, 90 to one another. The elongated sections 78, 96 are mounted together using the hole 100 in the second half 96 through which the screw passes and engages a corresponding cavity within the interior of the first half 76 to mount the halves to one another. Thereafter, the post 132 of the actuator 42, circumscribed by the spring 134, is inserted through the orifice 94 of the first half 76 so that the spring 134 abuts the floor 90, but the post 132 continues through the orifice 94 of the first half 76 and the corresponding orifice 108 of the second half 74. The cylinder 138 of the cam member 142 is inserted through the orifice 108 in the second half 74 to circumscribe the post 132. The diamond-shaped projection 72 includes a through hole extending into the hollow cylinder 138. With the button 130 and the cam member 142 sandwiching the circular ends 80, 98, a screw is inserted through the hole and manipulated to engage a cavity within the post 132. Continued manipulation of the screw acts to draw the exposed end of the post 132 into contact with a circumferential flange at the base of the cylinder 138, so that coaxial movement of the button 130 results in coaxial movement of the cam member 142, and vice versa.

Assembly of the extraction vessel holder 18 includes positioning the diamond-shaped projection 72 of the cam member 142 into the area defined by the diamond-shaped raised wall 66. It should be noted that the diamond-shaped projection 72 of the first cam member 142 is a mirror image of the diamond-shaped projection 72 of the second cam member 142. Also, the two diamond shaped raised walls 66 of the second part 28 of the handle portion 20 are mirror images of one another. The rounded corners of the diamond-shaped projection 72 are aligned with the rounded corners 70 of the diamond-shaped raised wall 66, thereby aligning the perpendicular corners of the diamond-shaped projection 72 with the perpendicular corners 68 of the diamond-shaped raised wall 66, so that each diamond shaped projection 72 is received within a corresponding cavity defined by the diamond-shaped raised wall 66. This arrangement rotationally locks the projection 72 in place with respect to the raised wall 66. The first part 26 is brought into contact with the second part 28 to sandwich the circular ends 80, 98 of each repositionable arm 22, thereby allowing the buttons 130 to protrude through the two orifices 38, 40. Five screws are inserted through corresponding holes 56 within the exterior of the second part 28, which are received within corresponding channels of the receivers to mount the first and second parts 26, 28 to each other. In this assembled form, the corresponding grooves 44, 46 cooperate to allow for a ninety degree range of movement for each repositionable arms 22 to pivot between a parallel position and a perpendicular position.

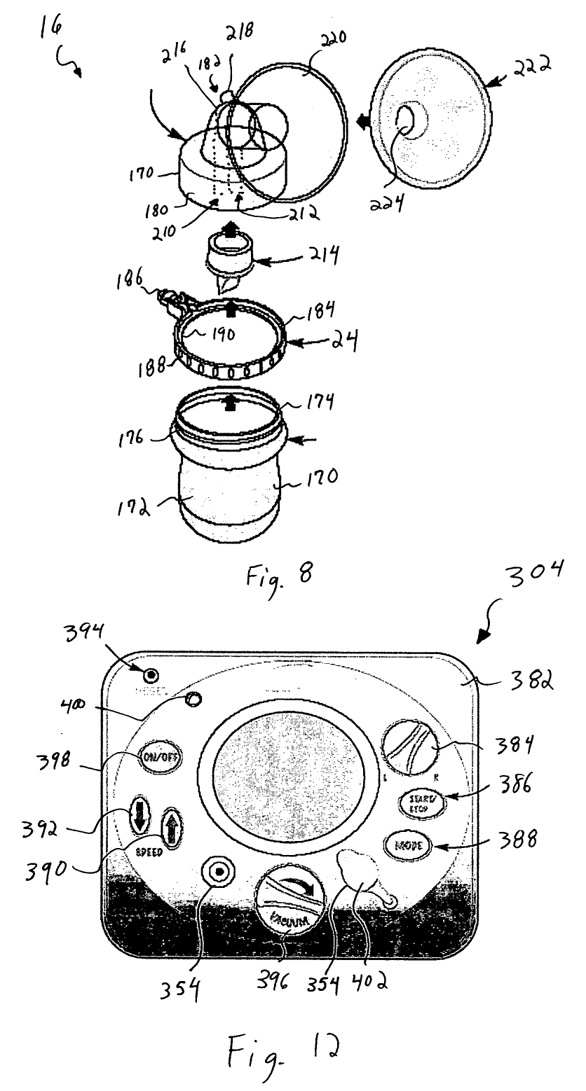

Referencing FIG. 8, an exemplary extraction assembly 16 includes an extraction vessel 170 mounted to a retention ring 24. The extraction vessel 170 includes a cup 172 having a circumferential flange 174 extending outward around the cup 172 and below a pair of spiral threads 176. The spiral threads 176 are received within corresponding grooves within a lid 180 of a horn 182. It should also be noted that the spiral threads 176 of the cup are also adapted to engage a bottle cap having a nipple for use with dispending the extracted milk from within the cup to a baby.

The retention ring 24 includes a circular portion 184 having an integrated projection 186. The circular portion 184 includes a vertical wall 188 having an interior surface from which a circular ledge 190 extends. The integrated projection 186 includes a key 192 at the end thereof that is adapted to interface with the teeth 88 within a longitudinal groove 82 to mount the retention ring 24 to the handle portion 20. The key 192 includes a rectangular block end 196 transitioning into a disc 198. On the opposing side of the disc 198 is generally rectangular portion 200 having opposing sets of projections 202 adjacent to the disc 198. The projections 202 are adapted to be seated within corresponding gaps between adjacent teeth 88, thereby securing the retention ring 24 to the handle portion 20. It should be noted that the top and bottom of the retention ring 24 are symmetrical.

The horn 182 includes an internal hollow cylinder 206 having a chord divider 208 that provides two parallel channels 210, 212 within the cylinder. An elastomeric check valve 214 is mounted to the distal end of the cylinder 206, but in a manner that allows communication between the channels 210, 212 at the distal end of the cylinder 206. The first channel 210 extends upward into a dome 216 that caps one end of the cylinder 206. A nipple 218 extends from the dome 216 and provides communication between an environment external to the nipple and the first channel 210. The nipple 218 includes a first hollow conduit extending from the dome 216 that includes a through hole providing communication with the first channel 210. The nipple 218 also includes a second hollow conduit that is concentric with the first conduit so that one end of the tubing 14 is adapted to be inserted into the circumferential space between the conduits, thereby allowing the interior of the tubing 14 to be in direct communication with the interior of the first hollow conduit of the nipple 218.

The second channel 212 of the horn 182 extends upward from the distal end of the cylinder 206 and into a funnel 220. The larger diameter end of the funnel 220 is adapted to receive a funnel insert 222 comprising an elastomeric material that is adapted to abut a woman's breast so that the orifice of the breast is surrounded by the funnel insert. The base of the funnel insert 220 includes a hole 224 providing communication between the funnel insert 220 and the second channel 212. Preferably, a circumferential seal is formed between the funnel insert 220 and the woman's breast, thereby drawing milk from the breast as suction is applied through the tubing 14 by way of the first channel 210. Thus, as suction is applied to the breast, milk is drawn from the breast, through the hole 224 in the funnel insert 220, through the second channel 212 and into contact with the check valve 214. Gravitational forces allow the check valve 214 to open when milk comes in contact with the valve 214, however, the elastomeric nature of the valve 214 closes the valve when no milk is flowing therethrough.

Referring to FIGS. 1-8, construction of the exemplary extraction assembly 16 includes positioning the retention ring 24 so that the circular portion 184 at least partially circumscribes the threads 176 of the cup. The circular ledge 190 of the retention ring 24 is brought into contact with the circumferential flange 174 of the cup 172. The cup 172, with the retention ring 24 in place, is brought into an engagement position so that the spiral threads 176 of the cup engage corresponding grooves within the lid 180 of the horn 182 to mount the cup to the horn, thereby sandwiching the retention ring 24. Twisting of the cup and/or lid in a clockwise direction couples the horn 182 to the cup 172 by frictional engagement, thereby inhibiting the retention ring 24 from rotation about either the cup 172 or horn 182 when the cup is securely mounted to the horn. Therefore, the orientation of the retention ring 24 with respect to the horn 182 should be established prior to completely twisting the cup 172 and horn 182 into locking engagement. It is preferred that the funnel 220 be facing away from the key 192 so that the funnel 220 will be facing the woman's breast, while the key 192 will be facing the handle portion 20. After the extraction assembly 16 is constructed, it may be mounted to the handle portion 20.

Two of the extraction assemblies 16 can be mounted to the handle portion 20 concurrently, or only one of the extraction assemblies 16 may be mounted to the handle portion 20. As discussed previously, the handle portion 20 includes two repositionable arms 22 that pivot between a parallel position where the arms are free to pivot (unlocked) and a perpendicular position where the arms are inhibited from pivoting (locked) to the parallel position. It is presumed that one or both of the arms 22 is moved to the locked position, prior to mounting the extraction assemblies 16, however, this is not a prerequisite as the extraction assemblies 16 can also be mounted to the repositionable arms 22 in the parallel position.

To mount an extraction assembly 16 to a repositionable arm 22, the key 192 of the retention ring 24 is inserted through the circular opening 84 of the repositionable arm 22 so that the rectangular block end 196 is received between the raised rails 126 that extend longitudinally along the interior of the arm 22. After the key 192 has been fully inserted into the opening 84, the extraction assembly 16 is repositioned along the length of the longitudinal groove 82 so that a pair of gaps on adjacent sides of one of the teeth 88 is occupied by the two projections 202 of the key. This likewise occurs on the opposing side of the key 192 so that two teeth 88 on opposing rows sandwich the key 192, thereby retaining the extraction assembly 16 with respect to the repositionable arm 22. To laterally reposition the extraction assembly 16 with respect to the arm 22, the extraction assembly 16 is slid along the longitudinal groove 82 and seated within another pair of gaps on adjacent sides of another one of the teeth 88. Removal of the extraction assembly 16 may be accomplished by laterally sliding the extraction assembly 16 until a portion of the key 192 of the retention ring 24 occupies the circular opening 84, so that the key 192 can be withdrawn through the opening 84.

Repositioning of the arms 22 between a parallel position (See FIG. 7) and a perpendicular position (See FIG. 5) is accomplished in one of two ways. The first process for moving the arm 22 from the parallel position to the perpendicular position simply requires manipulation of the arm 22 in a pivoting manner until the actuator 42 locks the arm 22 in the perpendicular position. This locking action is accomplished by the cam projections 144 of the cam member 142 becoming seated within a cavity 226 between the hourglass portion 112. The cam member 142 is biased by the spring 134, thereby pulling the cam projections 144 into the cavity when no opposite force is applied to the button 130. To reposition the arm 22 from the perpendicular position to the parallel position, the button 130 is pushed inward, thereby overcoming the bias of the spring 134 to force the cam projections 144 from the cavity 226. Concurrent with the button 130 being pushed inward to displace the cam projections 144 from the cavity 226, the arm 22 is pivoted so that the cam projections 144 ride upon the hourglass portion 112 thereby allowing the arm 22 to be completely pivoted to the parallel position.

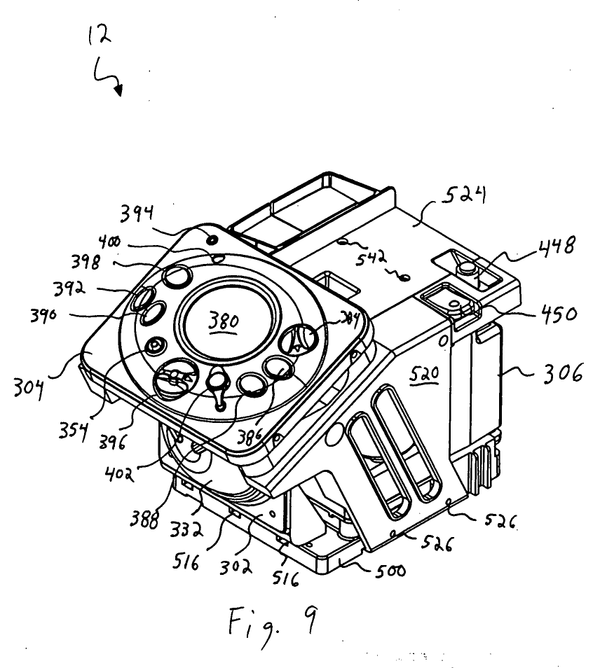

Referring to FIGS. 9-12, an exemplary breast pump 12 includes a number of interconnected modules 300, 302, 304, 306 mounted to a common frame 309. The first module is a motor module 300, and includes an electric motor 308 mounted to a gear housing 310. The gear housing 310 contains a series of gears mounted to the housing that are interconnected to the rotating shaft of the electric motor and concurrently interconnected to a driveshaft 320 exiting the housing. The drive shaft 320 is connected to an oblong arm 322, which is in turn connected to a linear extension arm 324 of the diaphragm module 302. In this configuration, rotational movement of the electric motor 308 is converted into reciprocating motion of the linear extension arm 324.

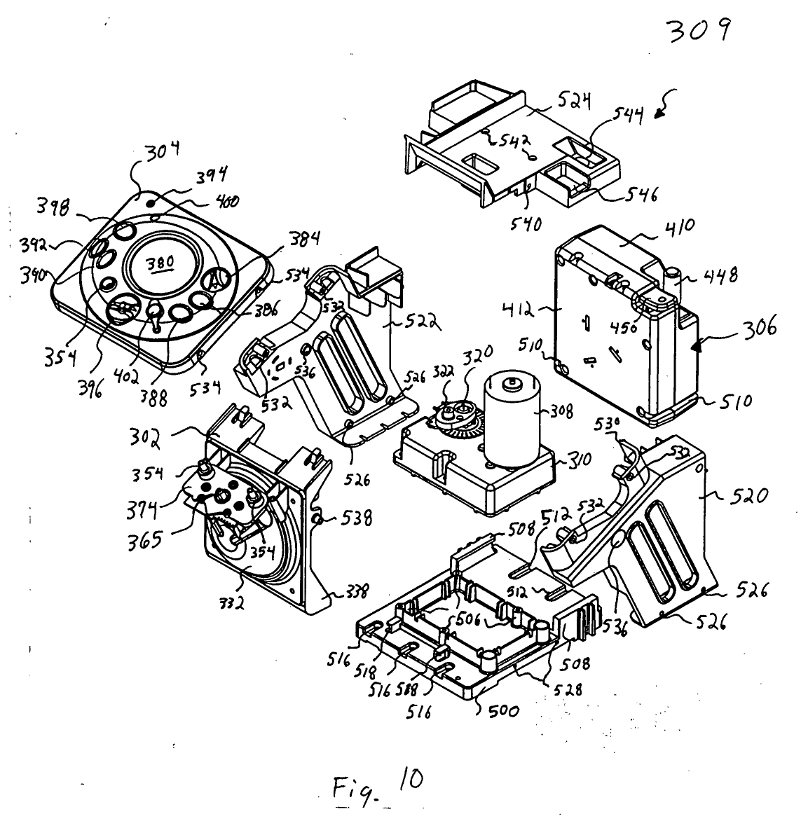

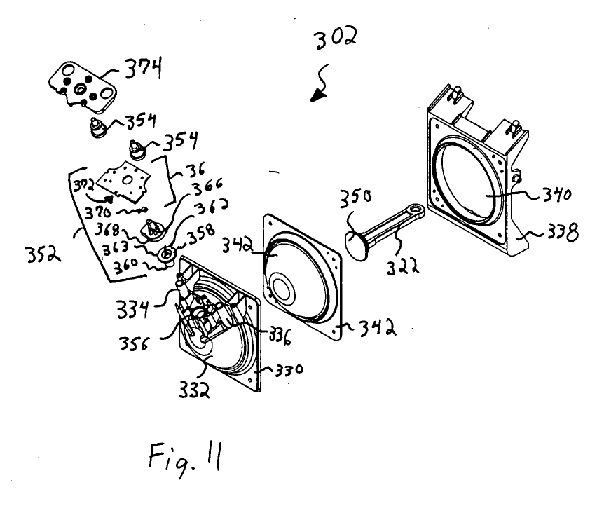

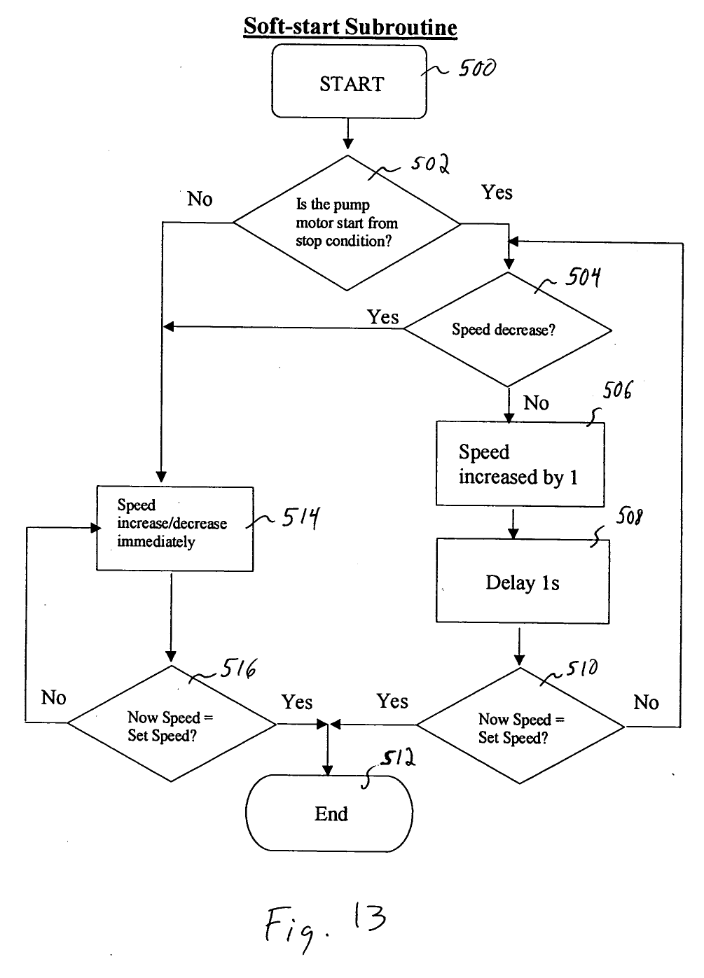

Referencing FIGS. 10 and 11, the diaphragm module 302 includes a domed housing 330 having a semispherical concavity 332 with two ports 334, 336 that are in communication with the control module 304 discussed below. A supplemental housing 338 is mounted to the domed housing 330 and includes a circular orifice 340 that shadows the semispherical concavity 332. An elastomeric semispherical diaphragm 342 is sandwiched circumferentially between the domed housing 330 and the supplemental housing 338. An interior side of the semispherical diaphragm 342 includes a convex dome having a frustoconical cavity therein that does not extend completely through the semispherical diaphragm 342. A frustoconical end 350 of the linear extension arm 324 is adapted to be seated within the frustoconical cavity of the semispherical diaphragm 342 and mounted thereto by way of a compression fit. This compression fit between the frustoconical cavity and frustoconical end 350 of the linear extension arm 324 may be replaced by a frictional fit using an adhesive or may be supplemented by using an adhesive. In this manner, reciprocating motion of the linear extension arm 324, as controlled by the control module 304, is operative to reposition the semispherical diaphragm 342 between a convex position (where the diaphragm 342 substantially abuts the semispherical concavity 332 as shown in FIG. X) and a concave position (where the diaphragm 342 is substantially withdrawn from the semispherical concavity 332).

The diaphragm module 302 also includes vacuum adjustment assembly 352 and tubing interconnectors 354. The vacuum adjustment assembly 352 allows a user of the breast pump to vary the suction applied to her breast. A vent hole 356 within domed housing 330 receives a plug 358 of vacuum adjustment assembly 352. The plug 358 includes a hollow cylindrical portion 360 having a perpendicular flange 362. The interior of the cylindrical portion 360 is occupied by a solid cylinder 363 having its own circumferential flange that substantially circumferentially contacts the interior walls of the cylindrical portion 360. The circumferential flange of the solid cylinder 363 includes a circumferential opening that increases in area in a gradual, circumferential manner from zero to a maximum size opening. The circumferential flange of the solid cylinder 363 interfaces with a hollow cylinder 365 of a valve 364. The hollow cylinder of the valve 364 includes a semicircular groove running axially along the exterior thereof. This groove extends to communicate with a hole 366 through a platform 368 that is in open communication with the external environment. The valve 364 is rotatably repositionable about the solid cylinder 363 of the plug 358 so that the groove within the hollow cylinder may be in communication with the circumferential flange of the solid cylinder 362. The groove can be shut off from communication with the interior of the semispherical concavity 332 or can be in direct communication with the semispherical concavity 332 simply by rotating the valve 364 to change the orientation of the groove with respect to the circumferential opening of the plug 358. For a user to decrease the suction applied, one simple needs to rotate the valve 364 so that the groove overlaps more of the circumferential opening of the plug 358. To increase the suction applied, one simple needs to rotate the valve 364 so that the groove overlaps less of the circumferential opening of the plug 358.

The vacuum adjustment assembly 352 also includes a speed adjustment component 367 comprising a repositionable element 370 and a contact pad 372. The repositionable element 370 includes two metal prongs that are interconnected to one another so that electrical contact with a first of the prongs will allow electrical contact with the second of the prongs. The repositionable element 370 is mounted to the valve 364 so that as the valve rotates, so too does the repositionable element. An underside of the contact pad 372 includes two semicircular, concentric conductive pads. The first pad is continuous, while the second pad is incrementally broken down into five separate pads, with each of the five separate pads corresponding to a predetermined speed of the electrical motor 308, by way of a ribbon connection to the control module 304. As the user rotates the valve 364 to adjust the amount of suction applied, the first prong of the repositionable element 370 stays in contact with the first pad of the contact pad 372. The second prong of the repositionable element 370 contacts one of the five separate pads. In this manner, current applied to the first pad is conducted through the prongs and through one of the five separate contact pads, depending upon the rotational position of the valve 364. Therefore, as the user opens or closes the vent to the semispherical concavity 332, the rotational speed of the motor 308 may be likewise increased or decreased depending upon the predetermined speed associated with each of the five separate contact pads. A mounting plate 374 is adapted to sandwich and secure the components of the vacuum adjustment assembly 352 and tubing interconnectors 354 to the domed housing 330 using a plurality of screws.

Referring to FIGS. 9, 10, and 12, the control module 304 includes a liquid crystal display 380 surrounded by a control panel 382. The liquid crystal display 380 shows the time and date (in minutes and seconds) during which the exemplary breast pump 12 is currently being operated, as well as the usage for the day and for each individual use. The control panel 382 includes an adjustment dial 384 for manipulating the suction applied to the left and right tubing interconnectors 354. For example, the suction applied to the left breast, by way of tubing connected to the left interconnector, may be too great, whereas the suction applied to the right breast, by way of tubing connected to the right interconnector, may be too little. The control panel 382 includes written indicia of an “L” and an “R” beneath the dial 384 so that rotational adjustment of the dial 384 toward the “R” would increase the suction on the right interconnector 354 and would correspondingly decrease the suction applied to the left interconnector 354. Conversely, rotation of the dial 384 toward the “L” would increase the suction on the left interconnector 354 and would correspondingly decrease the suction applied to the right interconnector 354. Just beneath the adjustment dial 384 is the start/stop actuator 386.

The control panel 382 also includes a mode actuator 388 just below the start/stop actuator 386. The mode actuator 388 allows a user to toggle between a number of options and settings associated with the breast pump 12. By pressing and holding the mode actuator 388 for a predetermined period (i.e., at least 5 seconds), the user can enter the time and date setup mode that is visually displayed by the liquid crystal display 380. Two arrows, one down arrow actuator 390 and up arrow actuator 392 are positioned across the control panel 382 from the mode and start/stop actuators 386, 388. Once the user has pressed and held down the mode actuator 388, the user can set the current time and date. First, the hour to be set will begin flashing, at which point the user can adjust the hour by using the up and down arrow actuators 390, 392. After the hour has been set, the user would press the mode actuator 388 to set the minute reading, again using the up and down arrow actuators 390, 392. This process would be repeated to set the am/pm, month, day, and year. When the user is finished setting these time and date options, the start/stop actuator 386 is depressed to exit the time and date setting mode.

The control panel 382 can also be utilized to view the usage of the breast pump 12. To view the usage on the liquid crystal display 380, the user depresses the mode actuator 388 briefly to switch between viewing “TODAY'S USAGE” and “CURRENT USAGE.” TODAY'S USAGE refers to the total volume of milk pumped, while CURRENT USAGE refers to the volume of milk pumped at the current setting. Each of these measures is reset automatically every 24 hours, however, these measures may also be reset manually by depressing the reset actuator 394 located in the upper left-hand corner of the control panel 382. The liquid crystal display 380 may therefore be utilized to view current usage time, daily usage time, aggregate usage time, as well as, speed of the pump, current time and date, and the distribution of suction applied to each connected tube.

The control panel 382 further includes a vacuum adjust dial 396 mounted to the vacuum adjustment assembly 352 to rotationally reposition the valve 364. In this manner, the suction applied to the right and left interconnectors 354 can be adjusted. In addition, the control panel includes an on/off actuator 398 as well as an LED 400 positioned above the on/off actuator 398 that is illuminated when the breast pump 12 is turned on. The control panel 382 may also include a connector cap 402 to close the suction port associated with either the right or left interconnector 354 when not in use.

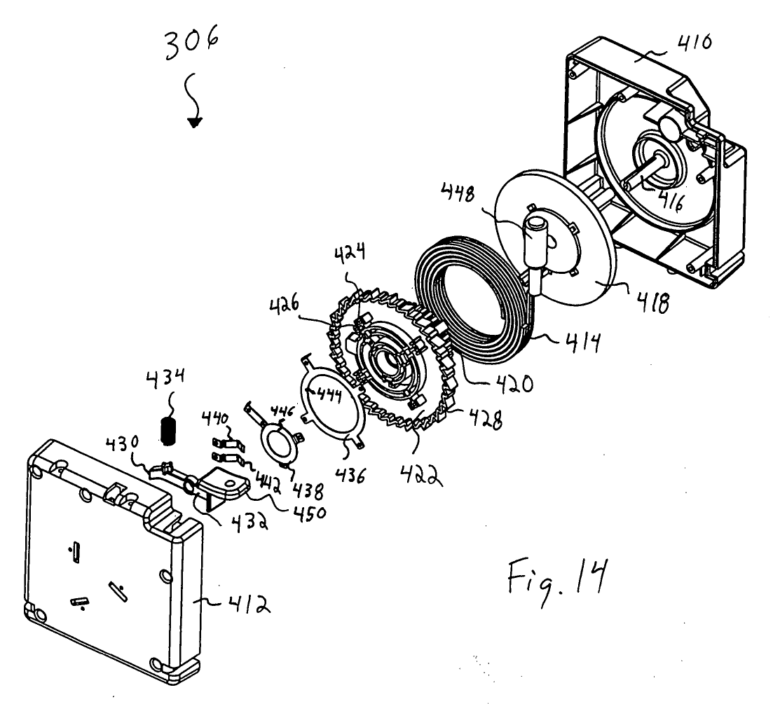

Referring to FIG. 13, the control module 304 is programmed to provide a soft start subroutine. A soft start subroutine ramps up the suction applied so that discomfort associated with an initial low pressure surge is reduced. An exemplary programming decision tree for the soft start routine begins with the step 500 of starting or powering on the breast pump 12. Thereafter, a determination 502 is made as to whether the pump motor is in preexisting motion or whether it is stopped. If the pump motor is stopped, a determination 504 follows whether the speed of the motor should be decreased. Obviously, if the motor is stopped the determination 504 will result in a “NO” answer, thereby resulting in a determination 506 that the speed of the motor should be increased by one incremental unit. A delay 508 follows the incremental speed increase, which is followed by a determination 510 as to whether the speed is equal to the maximum set speed. If the speed is equal to the maximum set speed, the routine is temporarily finished 512 and the rotational speed of the motor is maintained. If the determination 510 results in a finding that the speed is above or below the maximum set speed, then the routine restarts with a determination 504 as to whether the speed of the motor should be decreased. If this determination 504 concludes that the speed of the motor is over the maximum set speed, a determination 514 follows where the speed of the motor is determined and increased or decreased based upon a comparison with the maximum set speed. Thereafter, a determination 516 is made if the speed of the motor equals the maximum set speed. If the speed varies from the predetermined speed, the routine restarts with a determination 514 of the speed of the motor and thereafter increased or decreased based upon a comparison with the maximum set speed. Thereafter, a determination 516 is made if the speed of the motor equals the maximum set speed. This loop is continued until the speed of the motor equals the maximum set speed, thereby temporarily finishing 512 the loop and maintaining the speed of the motor. This soft start subroutine is reinitialized each time the breast pump 12 is started on the power is turned on after being off.

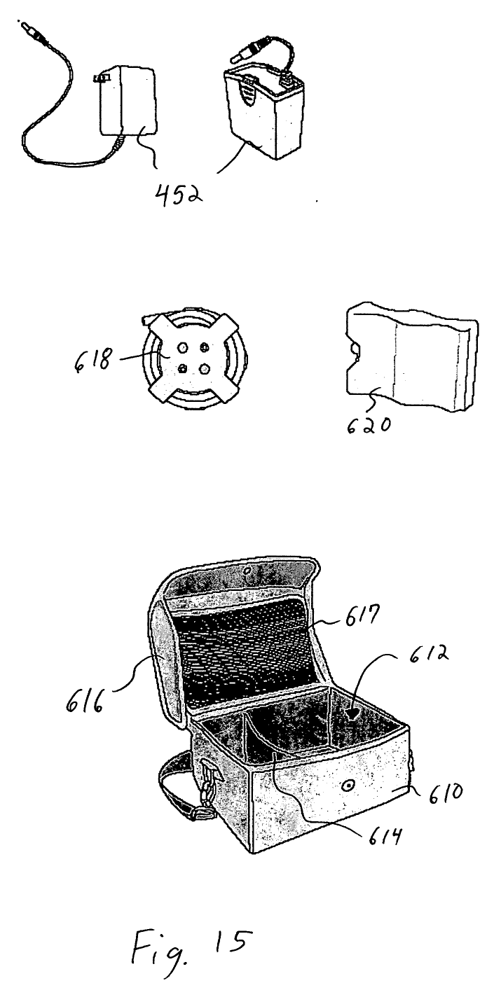

Referring to FIGS. 10 and 14, the power module 306 includes an outer housing consisting of complementary parts 410, 412 that define an internal cavity in which a retractable power cord 414 is stored. The first complimentary part 410 includes a perpendicularly extending post 416 adapted to receive a wire coil mounted to a rotatable disc 418. One end of the wire coil is mounted to the rotatable disc 418, while the opposing end of the wire coil interfaces with a catch on the interior of the first complimentary part 410. In this manner, as the disc 418 is rotated counterclockwise about the perpendicularly extending post 416, the coil exerts a biased force against the complimentary part 410 and the disc 418 attempting to rotate the disc 418 in a clockwise direction.

The backside of the disc 418 engages a drum 420 protruding from a wheel 422. The drum 420 includes the wire power cord 414 circumferentially disposed therearound and engages a pair of leads 424, 426 extending through the wheel 422. The wheel includes angled teeth 428 that are adapted to interface with a catch 430 on a pivoting arm 432 mounted to the second complimentary part 412. The arm 432 is biased by a spring 434 so that the catch 430 engages the angled teeth 428. A first conduction plate 436 is mounted to the backside of the wheel 422 using four screws so that the conduction plate 436 is in electrical communication with the first lead 424. A second conduction plate 438 is also mounted to the backside of the wheel 422 using four screws so that the plate 438 is in electrical communication with the second lead 426.

Two electrical prongs 440, 442 are mounted to the second complimentary part 412 and concurrently track the circular area 444, 446 exposed by each of the conduction plates 436, 438. A set of electrical lead lines extend from the second complimentary part 412 and are mounted to a power connection of the control module 304, while the opposing ends of the electrical lead lines are correspondingly in electrical communication with the prongs 440, 442.

To withdraw the cord 414 from inside of the complementary parts 410, 412, the end 448 of the cord, which is exposed outside of the internal cavity defined by the complementary parts 410, 412, is pulled on sufficient to rotate the wheel 422 and drum 420 in the counterclockwise direction. When the wheel 422 is rotated in the counterclockwise direction, the wire coil is twisted and exerts a biased force in the clockwise direction. The rotation of the wheel does not impede the electrical communication between the end of the cord and the wire leads extending from the second complementary part 412 because the prongs 440, 442 slide along a corresponding circular area 444, 446 exposed by each of the conduction plates 436, 438. Counterclockwise rotation of the wheel 422 allows the angled teeth 428 to pass beyond the catch 430 without the catch substantially impeding rotation in this direction.

To recoil the cord 414 inside of internal cavity defined by the complementary parts 410, 412, a tab 450 on the end of the arm 432 is depressed. This depression of the tab 450 overcomes the bias of the spring 434 to remove the catch 430 from the line of travel of the angled teeth 428. At this time, the bias exerted by the wire coil operates to rotate the wheel 422 and drum 420 in the clockwise direction, thereby winding the wire 414 around the drum until all but the end of the wire is drawn into the internal cavity. It should be noted that a preferred embodiment includes rotating the wire coil so that a clockwise rotational bias will be exerted by the coil even when the wire 414 is fully recoiled.

The end 448 of the cord, which is exposed outside of the internal cavity defined by the complementary parts 410, 412, includes a female receptacle adapted to accommodate a male projection connected to a power supply. Exemplary power supplies include, without limitation, any source of electricity such as those obtained using AC/DC plug-in converters, battery packs, and cigarette lighter connections 452 (See FIG. 15).

Referring to FIGS. 9 and 10, the common frame 309 includes a base platform 500 to which the gear housing 310 is mounted. Four screws piercing four holes within the gear housing 310 are received within four corresponding integrated cavities 506 within the platform to removably mount the gear housing 310 to the platform 500. A pair of slide rails 508 at the rear of the platform 500 extend upward and inward. These rails 508 are adapted to be received within a groove 510 of the power module 306 so that sliding the rails 508 into the groove 510 to mount the power module 306 to the platform 500. The power module includes two protrusions that are adapted to be received within corresponding grooves 512 within the platform 500. Each groove 512 and protrusion includes through holes adapted to receive screws thereby coupling the power module 306 to the platform 500.

The front of the platform 500 includes a series of grooves 516 and guide rails 518 that are adapted to interface with corresponding projections of the diaphragm module 302. Each groove 516 includes a through hole adapted to be aligned and receive a screw to mount the diaphragm module 302 to the platform 500. The guide rails 518 provide supplemental stability by sandwiching raised ribs of the diaphragm module 302 between the rails 518 and platform surface.

The common frame 309 also includes two side platforms 520, 522 that are mounted to the platform 500 are adapted to be concurrently mounted to a common top platform 524. Each side platform includes two side holes 526 through which screws are inserted to engage corresponding holes 528 within the base platform 500 to mount the side platforms 520, 522 to the base platform 500. Each side platform includes an upstanding projection 530 that is adapted to at least partially be recessed under the control module 304 so that two holes 532 allow entry of screws that engage corresponding holes 534 within the control module 304 to mount side platforms 520, 522 to the control module 304. An additional hole 536 within each side platform 520, 522 circumscribes a hollow channel projection 538 extending from the side of the diaphragm module 302 that is adapted to accept a screw to mount the side platforms 520, 522 to the diaphragm module 302. Finally, the common top platform 524 is mounted to each side platform 520, 522 and the power module 306 using a plurality of screw holes 540, 542. The top platform includes two openings 544, 546 that are adapted to allow throughput of the end 448 of the power cord 414 and the tab 450 on the end of the arm 432 so that the power cord 414 may be retracted after use of the pump 12 has been completed.

Referring to FIG. 1, the exemplary breast pump system 10 may also include a carrying case 600 for the breast pump 12. The exemplary carrying case 600 includes a recessed bottom area that accommodates the breast pump 12 and is contoured to generally match the profile of the pump 12. A folding lid 602 associated with the carrying case 600 may include an interior pocket 604 that is adapted to house items such as AC/DC plug-in converters, battery packs, and cigarette lighter connections (see FIG. 15) to connect a power supply to the pump 12.

Referring to FIG. 15, the exemplary breast pump system 10 may further include a secondary carrying case 610 may be utilized to house and transport such items as the tubing 14, extraction assemblies 16, and the extraction vessel holder 18. The secondary carrying case 610 may include a bottom cavity 612 that is divided into two or more smaller cavities by way of fixed or repositionable walls 614. The case 610 may be fabricated to include insulating materials so that the bottom cavity 612 will be insulated upon the case being closed. Exemplary uses for an insulated cavity include, without limitation, insulating refrigerated breast milk. In addition, one may utilize refrigerated or frozen packs 620 to cool or maintain the temperature of the breast milk that may be stored within the case 610.

The secondary carrying case 610 includes a repositionable lid 616 having an interior pocket 617 that may house such items as the tubing 14, extraction assemblies 16, and the extraction vessel holder 18. Along these lines, it is further within the scope of the invention to include a spindle 618 around which unused tubing 14 can be stored, where the spindle is housed within the secondary carrying case 610.

Following from the above description and invention summaries, it should be apparent to those of ordinary skill in the art that, while the methods and apparatuses herein described constitute exemplary embodiments of the present invention, the invention contained herein is not limited to this precise embodiment and that changes may be made to such embodiments without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

What is claimed is:

1. A breast pump assembly comprising:
a breast milk caddy including:
a frame having an arm repositionable with respect to a handle between a folded position and an extended position, and
a milk extraction device repositionably mounted to the arm of the frame, the milk extraction device including complementary top and bottom components removably mounted to one another, the bottom component including a breast milk extraction vessel, and the top component including a breast cone having an inlet for receiving a woman's breast and an outlet in communication with the extraction vessel for storing extracted milk, the top component also including a conduit for delivering suction to the inlet of the breast cone;
a breast pump including:
a fluid pump including an inlet and an outlet, where the fluid pump generates suction at the inlet during operation of the fluid pump, and
a control system in electrical communication with the fluid pump for controlling operation of the fluid pump, the control system including a soft start logic to gradually increase the suction generated by the fluid pump; and
tubing mounted in fluid communication with the fluid pump and the milk extraction device for delivering the generated suction from the fluid pump to the conduit of the milk extraction device.

2. The breast pump assembly of claim 1, wherein:
the control system includes a control panel having a visual display for providing feedback regarding the operation of the breast pump; and
the control panel includes a power actuator for turning off and on the visual display, a RPM actuator for at least one of increasing and decreasing the repeatable movements of the pump, and an mode actuator for selecting between two or more modes of operation of the breast pump.

3. The breast pump assembly of claim 1, wherein the breast milk extraction device includes a check valve in concurrent communication with the conduit and the outlet of the breast cone to allow extracted milk to flow into the extraction vessel while concurrently allowing suction from the conduit to be supplied to the outlet of the breast cone.

4. The breast pump assembly of claim 1, wherein the extraction vessel includes a cup having an open top and circumferentially distributed spiral projections adapted to interface with corresponding spiral projections of the top component to mount the top component to the extraction vessel, or interface with corresponding spiral projections of a nippled lid to mount the extraction vessel to the nippled lid for delivery of the extracted milk to a child by way of a nipple on the lid.

5. The breast pump assembly of claim 1, wherein:
the fluid pump includes a diaphragm pump; and
the soft start logic gradually increases the repeatable movements of the diaphragm pump to increase the suction generated by the diaphragm pump.

6. The breast pump assembly of claim 1, further comprising a carrying case including an insulated cavity for storing one or more extraction vessels having extracted milk.

7. The breast pump assembly of claim 1, further comprising a tube storage spindle for storing the tubing when not in use.

8. The breast pump assembly of claim 1, further comprising a plurality of extraction devices and tubing linking each extraction device to the breast pump, wherein the breast pump control system includes a user input for distributing the suction supplied to two or more of the plurality of extraction devices.

9. The breast pump assembly of claim 1, wherein the control system includes a control panel having a visual display for displaying at least one of current usage time, daily usage time, aggregate usage time, speed of the fluid pump, current time and date, and the distribution of suction applied to each connected tubing.

10. A breast pump assembly comprising:
a fluid pump including an inlet and an outlet, where the fluid pump generates suction at the inlet during operation of the fluid pump, and
a control system in electrical communication with the fluid pump for controlling operation of the fluid pump, the control system including a soft start logic to gradually increase the suction generated by the fluid pump, and further including a graphical user interface for displaying information regarding operation of the fluid pump.

11. The breast pump assembly of claim 10, further comprising an extendible power cord adapted to interface with a power source for supplying power to at least one of the fluid pump and the control system.

12. The breast pump assembly of claim 11, wherein:
the extendible power cord is retractable about a biased spool;
the biased spool is in communication with a repositionable catch to inhibit rotation of the spool; and
the catch is positionable between a catch position and a free position, whereby the free position allows rotation of the spool and the catch position inhibits rotation of the spool.

13. A breast milking caddy comprising:
a milk extraction device that includes a breast horn for communicating with a female breast and extraction vessel into which milk extracted from the female breast is deposited;
a handle; and
two repositionable arms mounted to the handle, each repositionable arm being repositionable between an extended position and a collapsed position, where each repositionable arm is configured to releasably engage the milk extraction device to orient the device in a retained position when the device is used to extract milk from the female breast and allow disengagement of the device subsequent to extraction.

14. The breast milking caddy of claim 13, wherein each arm is pivotally repositionable with respect to the handle so that the extended position orients the arm generally perpendicular to the handle, and where the collapsed position orients the arm generally parallel to the handle.

15. The breast milking caddy of claim 13, wherein:
each arm includes an actuator operative to cooperate with a corresponding feature of the handle to allow the arm to be positioned from the extended position to the collapsed position; and
the actuator includes a cam riding upon a camming surface of the arm operative to impart ratcheting action when the actuator is in the engaged position and allow free movement when the actuator is in a disengaged position.

16. The breast milking caddy of claim 15, wherein:
each arm includes a through hole that receives a projection of the actuator, the projection includes a button on one end and a camming body on the opposing end, the camming body including the cam having a sloped, arcuate shape that engages at least one of a plateau and depression of the arm, so that the cam is received within the depression in the engaged position, while the cam rides upon the plateau when the actuator is in the disengaged position, where the cam is biased to retain the cam within the depression when the arm is in the extended position, and where the bias must be overcome to reposition the arm from the extended position to the collapsed position; and
the corresponding feature of the handle engages the camming body to inhibit rotation of the camming body about the handle, thereby ensuring that rotation of the arm occurs around the actuator.

17. The breast milking caddy of claim 13, wherein each repositionable arm includes a plurality of predefined notches distributed along a length of thereof for receiving a retainer associated with the milk extraction device to mount the milk extraction device to the repositionable arm, where the milk extraction device can be repositioned along the length of the arm by varying which of the plurality of notches receive the retainer.

18. The breast milking caddy of claim 17, wherein:
the plurality of notches are internally disposed within each arm along a longitudinal groove; and
the retainer includes a circular ring and a perpendicular projection received by at least one of the plurality of notches, the retainer allowing the milk extraction device to be rotated with respect to the repositionable arm while the extraction device is mounted to the repositionable arm.

19. A breast milk storage vessel holder comprising a handle including first and second arms mounted thereto, the first and second arms being repositionable between a folded position and a locked, extended position, the first and second arms each including a longitudinal opening for receiving a peg of a circular bracket, the peg including projections received within spaced apart grooves on the inside of either the first or second arm, the circular bracket interposes first and second complementary parts of a milk extraction device, where a first complementary part includes a breast horn and a second complementary part includes a breast milk extraction vessel.

20. A breast milk storage vessel holder of claim 19, wherein at least one of the first arm and the second arm includes an actuator for repositioning the arm between the locked, extended position and the folded position.
